(19) United States
(12) Patent Application Publication
(10) Pub. No.: US 2008/0255447 A1
(43) Pub. Date: Oct. 16, 2008

(54) DIAGNOSTIC CATHETER

(76) Inventors: Henry Bourang, Irvine, CA (US); William R. Wertenberg, Newport Beach, CA (US); Ronald J. Hosmer, Mission Viejo, CA (US); Christopher Buller, Vancouver (CA); Duy Nguyen, Corona, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/735,722

(22) Filed: Apr. 16, 2007

(51) Int. Cl. A61M 25/01

(52) U.S. Cl. 600/434

(57) ABSTRACT

A diagnostic catheter having both a guidewire lumen and at least one contrast lumen is disclosed. In one embodiment, the contrast lumens allow the user to deliver contrast to a target area within the patient without removing the guidewire. Preferably, a plurality of contrast exit ports are included along the length of the catheter body to evenly and consistently distribute contrast into the blood stream, thereby providing improved visualization during radioscopy.

### FIELD OF THE INVENTION

The present invention relates to medical devices and, more particularly, to an improved diagnostic catheter and method of use.

### BACKGROUND

Catheters are used in a number of medical procedures within various conduits of the body for the purposes of diagnosis and treatment. Typically, catheters have an elongated body with at least one interior lumen which is sized to accept a guidewire. The guidewire is usually inserted first into a patient so that a distal end of the guidewire is positioned at or near a desired target location within the patient. The catheter lumen is fed on to the guidewire, allowing a distal end of the catheter to advance to the target location within the patient.

For example, angioplasty generally includes the steps of inserting a guidewire through a vascular access needle into the femoral or jugular artery or vein and manipulating the external proximal end of the guidewire to advance the distal end of the guidewire through the patient's arterial tree to a predetermined vascular destination.

Achieving a desired location within a patient can be difficult to attain by the feel of the catheter alone and therefore it is often necessary to visualize the anatomical shape of the vessel. One common visualization technique involves injecting contrast fluid into the vessel. A material in the contrast fluid, such as iodine, is visible to X-rays or other radiation and thereby allows the user to visualize the nearby vascular structure. Since the guidewire and the catheter often include radiopaque portions, these devices are also seen relative to the contrast-filled vascular structures. In this respect, the user can visualize the position of their guidewire or catheter relative to the patient's vascular structure and determine if the desired target location has been achieved.

To introduce contrast into the vessel, the guidewire is typically retracted and removed from the lumen of the catheter, allowing contrast fluid to be injected through the lumen of the catheter. The removal of the guidewire prolongs the procedure which increases the risk of complication to the patient. Furthermore, exchanging the guidewire may lead to a loss of the target position at the distal end of the catheter and therefore require additional searching and repositioning. In some procedures it is not possible to reintroduce the guidewire due to spasms of vascular muscles at the target location. In procedures where reintroduction is possible, additional trauma may result at the target location.

Additional disadvantages are also present with diagnostic catheters depending on the location being accessed. For example, existing coronary sinus illumination is currently accomplished by injecting a contrast medium within a single lumen delivery catheter which is placed at or near the ostium of the coronary sinus. Since the injection is made in the opposite direction of the blood flow, the infusion of contrast using this delivery method sometimes results in a resolution that may not be adequate. In this respect, current visualization systems often fail to adequately identify anatomical obstacles within the coronary sinus such as prominent valves near the coronary sinus ostium.

Current diagnostic catheters also lack more specialized functionality such as the ability to cannulate deep within the coronary sinus, for example during a procedure to treat heart valve regurgitation. Currently, the coronary sinus is accessed using angiographic catheters (e.g., 5 or 6 Fr Judkins Right JR4 coronary aniography catheter) to position the tip of the catheter at the ostium of the coronary sinus. The limitations of these devices are that since its intended use is for angiographic purposes, the tip curvature is not ideal to access the coronary sinus and thus make it difficult to cannulate. After locating the ostium, a guidewire is inserted into the diagnostic catheter from the proximal end and used to track to the great cardiac vein by manipulating (e.g., via torquing) the proximal end of the guidewire. The challenge with this technique lies with the difficulty of directing the guidewire tip into a desired side branch within the coronary sinus without causing the guidewire to become stuck or hung up. Current angiographic catheter tips are unable to advance deep within the coronary sinus due to their necessary rigidity. Thus, after the guidewire is positioned within the great cardiac vein, the angiographic catheter is withdrawn and exchanged for the over-the-wire guide/dilator catheter. The dilator is withdrawn when the guide catheter reaches a position at the ostium. This method of exchange increases procedural times and possible risk or errors. Additionally, when a guidewire is utilized to navigate deep within the coronary sinus, there is a risk for soft tissue damage such as tearing or piercing of the coronary sinus.

Thus, there is a need to provide an improved diagnostic catheter that is easier to use, reduces the number of guidewire exchanges during a procedure, decreases the likelihood of causing trauma to the patient and provides improved contrast visualization.

### OBJECTS AND SUMMARY OF THE INVENTION

It is an object of the present invention to provide an improved diagnostic catheter that overcomes the limitations of the prior art.

It is a further object of the present invention to provide a diagnostic catheter that decreases the likelihood of causing trauma to the patient during a procedure.

It is yet another object of the present invention to provide a diagnostic catheter that provides improved contrast visualization over prior art catheters.

An aspect of some preferred embodiments of the present invention relates to a diagnostic catheter having both a guidewire lumen and at least one contrast lumen. These lumens allow the user to deliver contrast to a target area within the patient without removing the guidewire.

In another aspect of some preferred embodiments of the present invention, multiple contrast lumens are included within the diagnostic catheter, maintaining structural integrity and kink resistance within the catheter body while providing a consistent flow of contrast to a target location.

In yet another aspect of some preferred embodiments of the present invention, a plurality of contrast exit ports are included along the length of the catheter body to more evenly and consistently distribute contrast into the blood stream, thereby providing improved visualization during radioscopy.

### BRIEF DESCRIPTION OF THE DRAWINGS

FIG. 1 illustrates a cross sectional side view of a preferred embodiment of a diagnostic catheter according to the present invention;

FIG. 2 illustrates a cross sectional view of the diagnostic catheter of FIG. 1 taken along lines 2-2;

FIG. 3 illustrates a cross sectional side view of a preferred embodiment of a diagnostic catheter according to the present invention;

FIG. 4 illustrates a cross sectional side view of a preferred embodiment of a diagnostic catheter according to the present invention;

FIG. 5 illustrates a cross sectional view of the diagnostic catheter of FIG. 1 taken along lines 5-5;

FIG. 6A illustrates a side view of a preferred embodiment of a diagnostic catheter according to the present invention;

FIG. 6B illustrates a side view of a preferred embodiment of a diagnostic catheter with balloons according to the present invention;

FIGS. 6C and 6D illustrate cross sectional views of the catheter of FIG. 6B;

FIG. 6E illustrates a perspective view of a preferred embodiment of a diagnostic catheter with an occlusion balloon according to the present invention

FIG. 7 illustrates a side view of a preferred embodiment of a diagnostic catheter according to the present invention;

FIG. 8A illustrates a side view of a preferred embodiment of a diagnostic catheter and a guide catheter according to the present invention;

FIGS. 8B and 8C illustrate cross sectional views of the diagnostic catheter of FIG. 8A;

FIG. 9 illustrates a side view of preferred embodiments of a diagnostic catheters and a guide catheter according to the present invention; and

FIGS. 10 and 11 illustrate a partial cross sectional view of a heart with a preferred embodiment of diagnostic catheter according to the present invention,

### DETAILED DESCRIPTION OF THE INVENTION

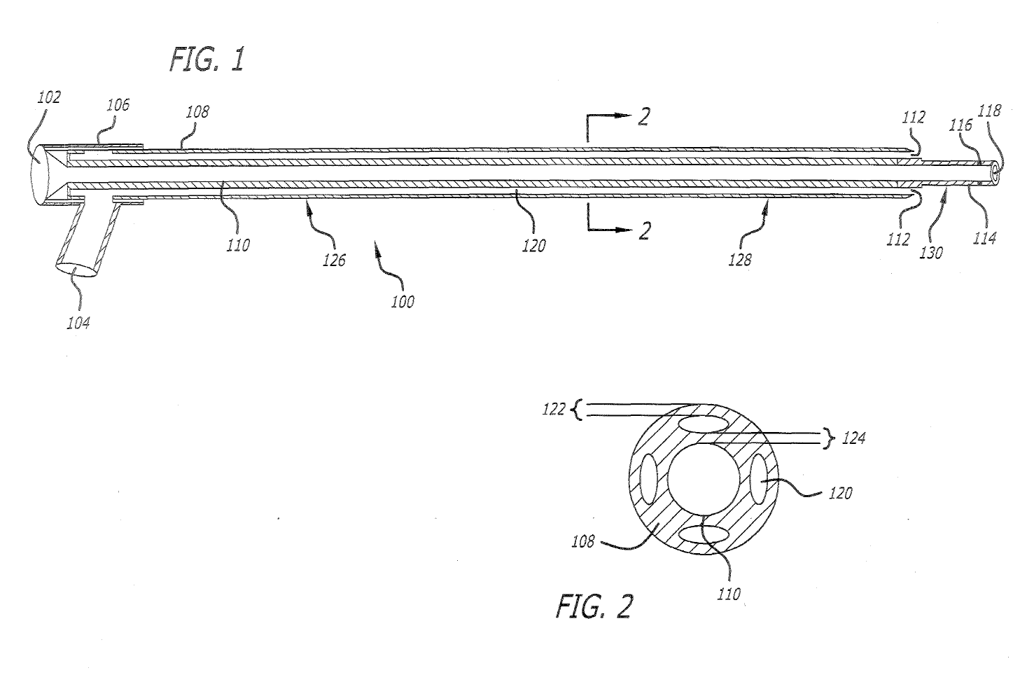

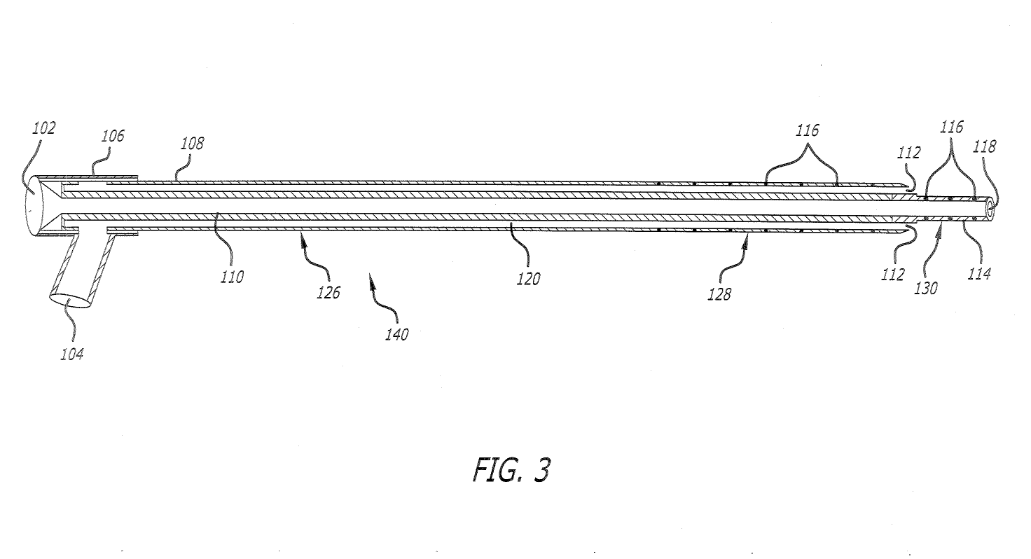

FIGS. 1 and 2 illustrate a preferred embodiment of a diagnostic catheter 100 according to the present invention. The diagnostic catheter 100 includes both a guidewire lumen 110 and four contrast lumens 120 which allow the user to deliver contrast to a target location within a patient without removing the guidewire.

The catheter 100 has a generally elongated body 108 that includes regions 126, 128 and 130. Preferably, each region 126, 128 and 130 is progressively more flexible than the next, allowing regions 128 and 130 to better conform to the tortuous pathway of the patient's vascular system while minimizing any trauma cause by the distal end of the catheter 100. This feature is advantageous as compared with prior art measurement catheters which are more rigid and therefore may reshape the anatomy when advanced through the vascular system of a patient.

In one example, the region 126 is about 70 cm in length and composed of a high density polyethylene with a Durometer between about 58 and 72 Shore D, the region 128 is about 15 cm and composed of a 50/50 polyethylene having a Durometer between about 55 and 63 Shore D and the region 130 is about 5 cm and composed of a low density polyethylene with a Durometer between about 41-60 Shore D.

As best seen in the cross sectional view of FIG. 2, taken along line 2-2, the catheter 100 includes four contrast lumens 120 along the body 108. Returning to FIG. 1, each of the lumens 120 connect to a single injection port 104 in the hub 106 at the proximal end of the catheter 100. The distal end of the contrast lumens 120 terminate at exit ports 112 at the distal end of the catheter 100. In this respect, contrast is injected into the injection port 104 and passes distally through the contrast lumens 120 and into the vascular system of the patient through ports 112.

The catheter is generally configured and sized to minimize kinking of the contrast lumens while maintaining adequate contrast flow to the exit ports 112. In a more specific example of such a configuration, referring to FIG. 2, the catheter body 108 includes an outer diameter of about 0.061 inches, with the inner diameter of the guidewire lumen 110 being about 0.039 inches (to fit over a 0.035 inch diameter guidewire) and each contrast lumen 120 is positioned at a distance 122 of about 0.003 inches from the outer circumference of the body 108 and positioned at a distance 124 of about 0.003 inches from the guidewire lumen 110.

Referring to FIG. 1, the guidewire lumen 110 includes a proximal entry port 102 and a distal exit port 118 which allows the catheter 100 to advance over a guidewire (not shown). The exit port 118 extends distally past the contrast exit ports 112, creating a reduced diameter distal end 114 of the catheter 100. Preferably the interface between the larger diameter of the body 108 and the smaller diameter of the distal end includes an angled transition to minimize the abruptness of the transition and thereby provide a generally tapered shape. Such a tapered shape may allow a user to more easily maneuver the catheter 100 into vessels with relatively small passages. For example, this reduced diameter end 114 can have a length of about 1-5 cm with a 4 French diameter. Additionally, a radiopaque marker 116 is located at the distal end of the catheter 100 for viewing the catheter position during visualization techniques (e.g. radioscopy).

In operation, the user advances a guidewire into the vascular system of a patient until a distal end of the guidewire is thought to be positioned near a desired target location. The catheter 100 is then fed over the guidewire so that a proximal end of the guidewire passes into the exit port 118, through the guidewire lumen 110 and out the port 102.

When the user wishes to visualize the location of the guidewire and catheter 100 relative to the patient's vascular structure, contrast media is injected into the injection port 104, passing down the contrast lumens 120 and out the exit ports 112. Radioscopy is performed, exposing the patient to radiation, such as X-rays, to generate an image of the relative position of the guidewire and catheter 100 in the patient.

While the catheter 100 is illustrated with four contrast lumens 120, it should be understood that greater or fewer contrast lumens 120 may be included according to alternate preferred embodiments of the present invention. For example, the catheter 100 may include 2, 3, 4, 5 or 6 contrast lumens 120.

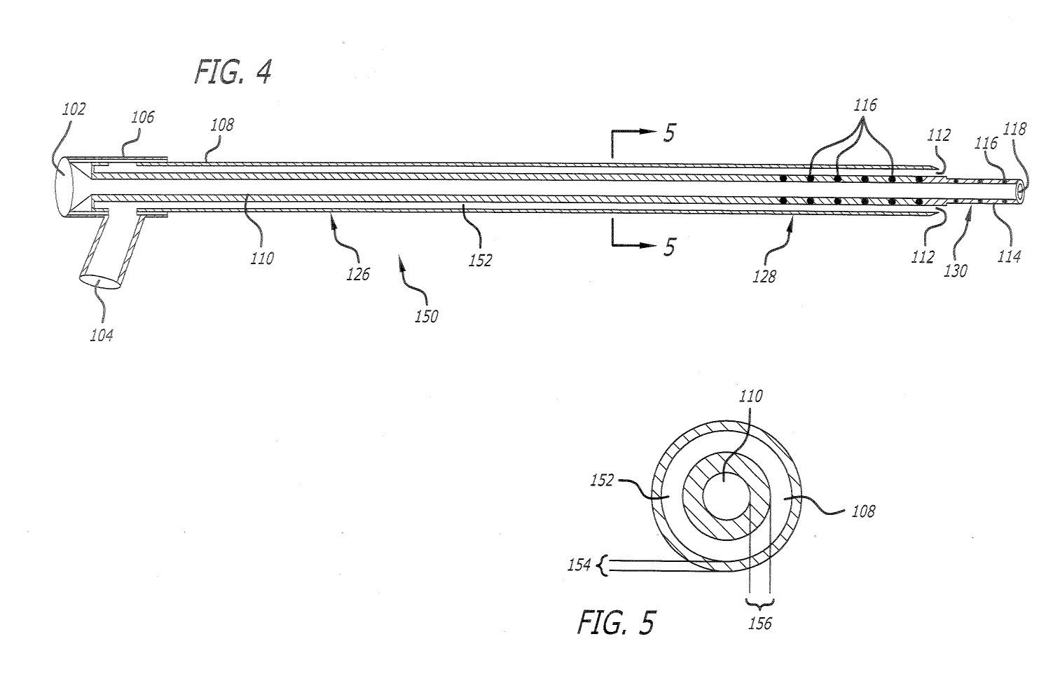

FIG. 3 illustrates another preferred embodiment of a diagnostic catheter 140 according to the present invention. The diagnostic catheter 140 is generally similar to the previously described catheter 100, including a body 108 having a central guidewire lumen 110 and four contrast lumens 120. However, the catheter 140 further comprises a plurality of radiopaque markers 116 located along the outer diameter of the body 108 and the reduced diameter distal end 114.

In some procedures, a measuring guidewire is used; having a plurality of radiopaque markers spaced at known intervals along its length. These markers allow the user to measure vascular features or positions within the patient during a visualization technique. In contrast, the present diagnostic catheter 140 integrates these measuring markers 116 into the body 108, allowing the user to make measurements during a visualization procedure while using an ordinary guidewire. In this respect, the user is free from removing the ordinary guidewire and replacing it with a measuring guidewire during a procedure. This reduces the trauma induced from the guidewire switch, reduces the time required for the procedure, and finally reduces the expense of the more expensive measuring guidewire.

The markers 116 are preferably embedded into the body 108, so as to have the same outer diameter as other portions of the body 108. In one example, the markers 116 are arranged along the length of the catheter in 1 cm increments along the distal end of the catheter 100 for about 25 cm. In a further example, the markers 116 have a band shape around the diameter of the body 108, a width between 0.001-0.002 inches, and a composition of platinum, iridium or gold.

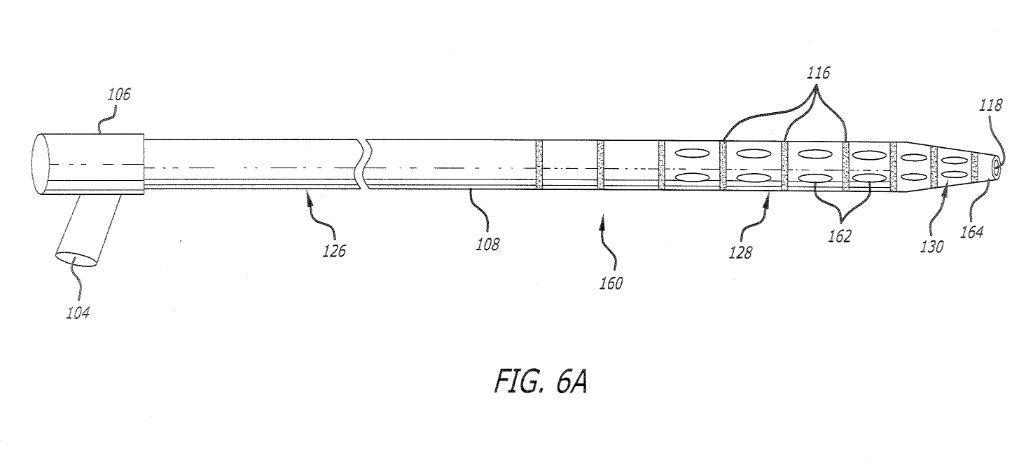

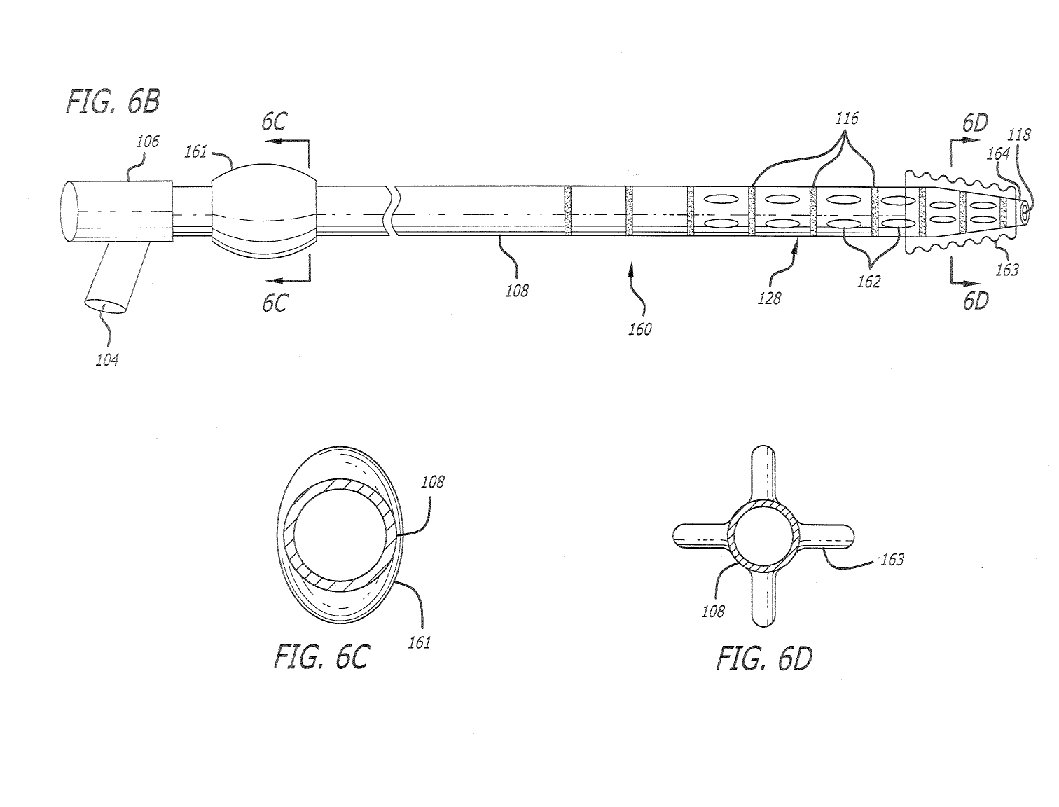

Referring to FIGS. 4 and 5, another preferred embodiment of a diagnostic catheter 150 is illustrated according to the present invention. This catheter 150 is generally similar to the catheters 100 and 140 of the previous preferred embodiments. However, the present catheter 150 includes a single axial contrast lumen 152 through the body 108. The coaxial contrast lumen 152 provides a lumen with an increased cross sectional area compared with the previously described preferred embodiments and therefore maximizes the amount of contrast media that can be delivered. Additional information regarding an axial lumen arrangement can be found in U.S. Publication No. 2005/0273077, the contents of which are hereby incorporated by reference.

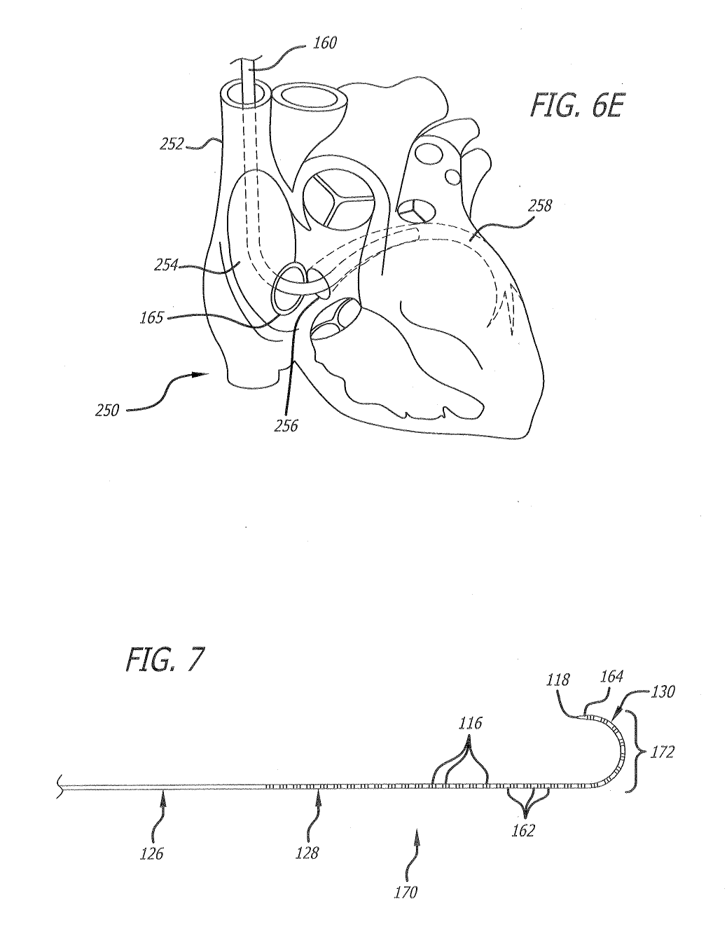

Referring now to FIG. 6A, another preferred embodiment of a diagnostic catheter 160 is illustrated according to the present invention. Again, this catheter 160 is generally similar to the previously described catheters 100, 140 and 150. However, instead of a single contrast exit port on the distal end of the catheter, a plurality of smaller diffusion ports 162 are provided along the length of the catheter body 108.

Current dispersion catheter technology typically pertains to delivery of drug therapy and dispersions of therapeutic solutions for pulse and slow infusion delivery. In contrast, the plurality of diffusion ports 162 more widely disperses contrast solution to aid in the identification of anatomical features, such as heart landmarks, vascular navigation, coronary sinus identification, cannulation, measurement and deep navigation. In a further example use, the improved contrast dispersion may improve identification of obstacles to coronary sinus cannulation such as prominent valves such as the thebesian, coronary venous and the valve of vieussen at the transition between the coronary sinus and great cardiac vein.

Preferably these diffusion ports 162 have a circular shape, oval shape, slit shape or any combination of the three. These ports 162 can be located at different positions around the circumference of the body 108 to achieve different contrast dispersion patterns. For example, the ports 162 may be located at 45, 60 or 120 degree intervals around the circumference of the body 108. Further, the sizes of the ports 162 may increase or decrease along the length of the catheter 160. Such a port diameter gradient may be configured to compensate for decreasing contrast pressure toward the distal end of the catheter 160 or configured to disperse different amounts of contrast along the length of the catheter 160. Additional delivery port information can be found in U.S. Publication No. 2006/0064011, the contents of which are hereby incorporated by reference.

In one example, the plurality of ports 162 are situated along a length of the catheter 160 of about 20 cm and have a port diameter of about 0.5 mm and are positioned at 120 degree intervals around the circumference of the body 108.

In addition to the plurality of radiopaque markers 116 placed at regular intervals along the catheter 160, the catheter body 108 may also include radiopaque graduations for further indicating length measurement during radioscopy. These graduations may be created, for example, by imbedding radiopaque markers within the body of the catheter, hot stamping radiopaque ink into the catheter body or impregnating regions such as the distal tip with a radiopaque compound. The atraumatic tip 164 of the catheter 160 may also include radiopaque materials to better define the distal end of the catheter 160 during a visualization procedure.

Optionally, the catheter 160 may include inflatable balloons to aid with navigation, dilation or provide diametric measurements (e.g., a balloon may be distended to a set diameter using a predetermined volume as a diametric estimator). Further, a balloon may be used as a positioning tool, for example to change the trajectory of a catheter and especially to “kick” the catheter to one side when a bifurcation is encountered.

Turning to the example of FIGS. 6B and 6D, the catheter 160 includes one or more inflatable balloons 163 disposed near the distal tip of the catheter 160 as a positioning tool. The balloons 163 may be used to change the trajectory of the catheter 160 by “kicking” or steering the catheter 160 to one side of a lumen when, for example, a bifurcation is encountered. Further, the balloons 163 may be used to aid in dilation of desired target areas. Preferably, the balloons 163 are attached to the body 108 by heat welds, bonds, or sutures/adhesives. As seen best in FIG. 6D, the balloons are arranged in a four-axis cross shape which allows the contrast to flow from the ports 162 around the catheter tip. Although balloons 163 are illustrated for purposes of discussion, it will be recognized that other types of suitable expandable members may be used instead of balloons.

In another example, FIGS. 6B and 6C illustrate an elliptical measurement balloon 161 for measuring an outer diameter of a lumen or structure within a patient. More specifically, the outer diameter of the balloon 161 is calibrated with respect to a known volume of filling fluid. When the catheter 160 is positioned within a patient, such as in the right atrium or coronary sinus, the user inflates the balloon 161 with fluid until the fluid injection encounters resistance. The amount of fluid injected into the balloon 161 therefore can be used to determine the diameter of the target lumen within the patient.

FIG. 6E illustrates another example of a circular occlusion balloon 165 on catheter 160. Preferably, the occlusion balloon 165 is mounted at least about 20 cm from the distal end of the catheter 160 for use in occluding a coronary sinus of a patient. The catheter 160 is preferably expanded in the right atrium 254 against the coronary sinus 258, partially or completely occluding blood flow. Thus, when contrast is injected from a distal portion of the catheter 160, the contrast remains in the coronary sinus 258 longer and thereby produces a better image of the patient's lumen structure. Preferably, the occlusion balloon 165 has a generally oval shape and is “floating” (i.e., the fluid lumen of the balloon 165 is positioned away from the catheter body, connected by non-inflating portions). Alternately, the occlusion balloon can be sized and shaped similar to the previously discussed balloon 161.

Additionally, the occlusion balloon 165 may be used as a reference point by contacting various structures within the patient. For example, when expanded, the occlusion balloon 165 may contact the ostium of the coronary sinus 258, thereby providing a tactile indication to the user that the catheter 160 has been fully inserted into the coronary sinus 258.

In alternative configurations, the occlusion balloon 165 may be configured to slide relative to the catheter 160 to adjust the balloon position relative to the distal end of the catheter 160 or to measure different features within the patient. For example, the occlusion balloon 165 can be attached to an outer sheath (not shown) that surrounds and therefore slides relative to the catheter 160.

Additionally, the body 108 of the catheter 160 may include braiding wire material (e.g., 304 or 316 grade stainless steel with wire diameter ranges of about 0.001-0.004 inches if rounded and 0.003-0.008 inches if flat wire) for improved torque control and pushability during insertion and manipulation of the catheter 160.

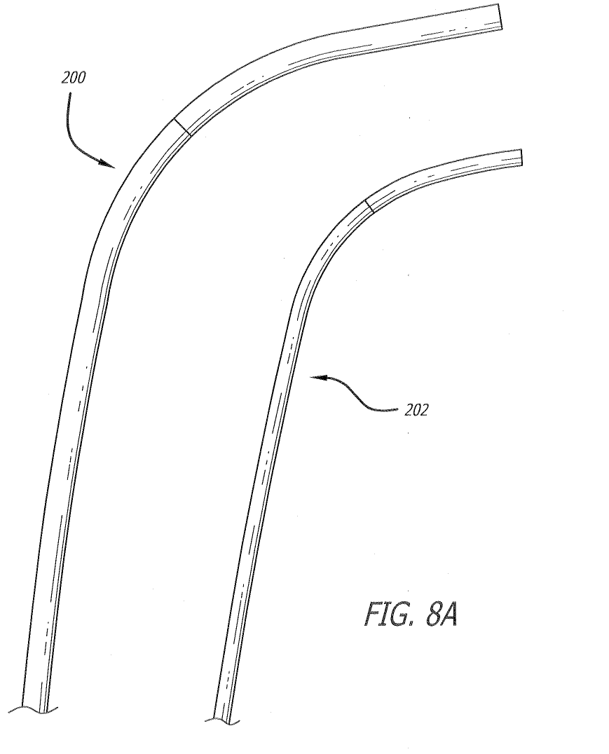

In another preferred embodiment seen in FIG. 7, a catheter 170 is provided which is similar to the previously described catheter 160. However, the current catheter 170 includes a curved distal end region 172 to assist a user in navigation and conformance to tight radius vessels.

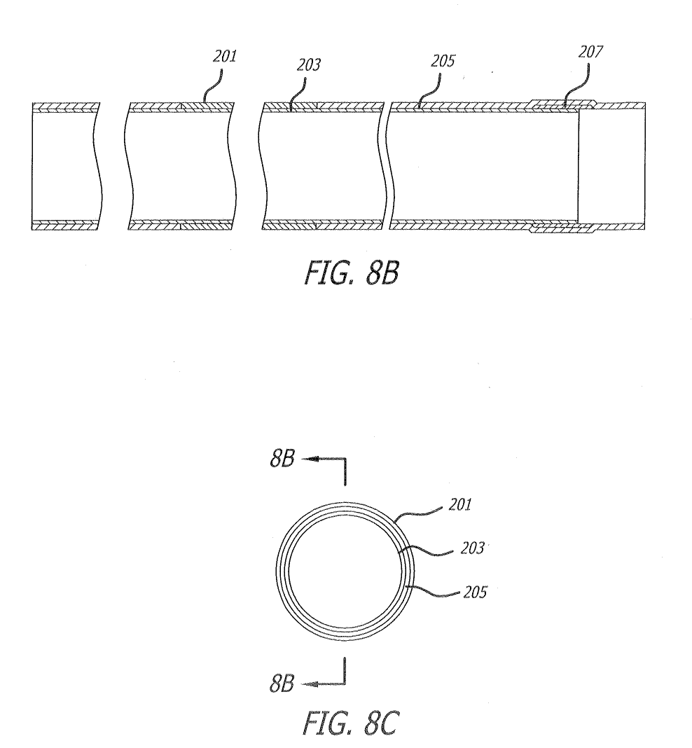

FIG. 8A illustrates another preferred embodiment of a diagnostic catheter 200 and a guide catheter 202 according to a preferred embodiment of the present invention for providing improved deep access to the coronary sinus. Previous diagnostic and guide catheters typically could not be advanced further than 1 cm into the coronary sinus. However, catheters 200 and 202 can achieve deep access to the coronary sinus (i.e., beyond the coronary sinus and great cardiac vein and into the anterior interventricular vein) by including regions of varying softness or flexibility near their distal ends and by including predetermined distal end curvature. In this respect, the curvature of the distal end encourages the catheters 200 and 202 to pass into and through the side branches of the coronary catheter while the increasing flexibility reduces the likelihood of causing damage during the procedure.

As seen in the cross sectional views of FIGS. 8B and 8C, the diagnostic catheter 200 preferably includes an outer polymeric layer 201 defining an exterior of the catheter 200, a wire braid layer 205, an inner polymeric layer 203 defining an interior lumen and a marker band 207. For example, the inner and outer layers may comprise polyethylene, polyurethane, polyester, PET, PEBAX, Hytrel or other biomedical grade polymers. Further, the inner layer may include lubricious material such as polyethylene (PE), PTFE or other medical grade polymers with a coefficient of friction of about 0.1 or less. In a more specific example, the outer polymeric layer 201 includes a distal section composed of 98% PEBAX 3533, a mid section composed of 98% PEBAX 5533 and a proximal section of 98% PEBAX 7233 and the inner polymeric layer 203 that is composed of PTFB. In this respect, the varying Durometer of each of the sections of the outer polymeric layer 201 allow for progressive flexibility towards the distal end of the catheter 200 during a procedure.

The wire braid layer 205 is included between the inner polymeric layer 203 and outer polymeric layers 201 of the diagnostic catheter 200, positioned along some or all of the catheter length. For example, the braided composite may be composed of polymeric filaments such as PET, Kevlar, Vectran or metallic wires and these wires preferably have a round diameter from about 0.0005-0.007 inches or a flat profile in the range of about 0.0005″×0.003″ to 0.003″×0.007″. The braid density can be increased to increase the rigidity of the catheter 200 (e.g., between about 30-80 picks per inch).

As previously indicated, the diagnostic catheter 200 has variable flexibility along its length so that the flexibility progressively increases towards the distal end of the catheter 200. For example, the diagnostic catheter 200 may have a distal segment with a firmness between about 25-72 Durometer shore hardness, an intermediate transition segment with a firmness between about 25-72 Durometer shore hardness (but greater than the distal segment), and a proximal segment with a firmness between about 40-72 Durometer shore hardness (greater than the intermediate transition segment).

The tips of the catheters 200 and 202 have a predetermined curve shape which allows a user to more easily direct the distal end of the catheter 200 and 202 into the side branches of the coronary sinus. Preferably, the curves of these angle ranges are between about 30-100 degrees. In one example, a catheter includes a distal region with a curve radius of about 1.50 inches across a 90 degree span. In another example, a catheter includes a distal curve having a radius of about 1.15 inches followed by an opposing intermediate curve having a radius of about 0.8 inches followed by an opposing curve having a radius of about 1.15 inches forming a generally question mark shape.

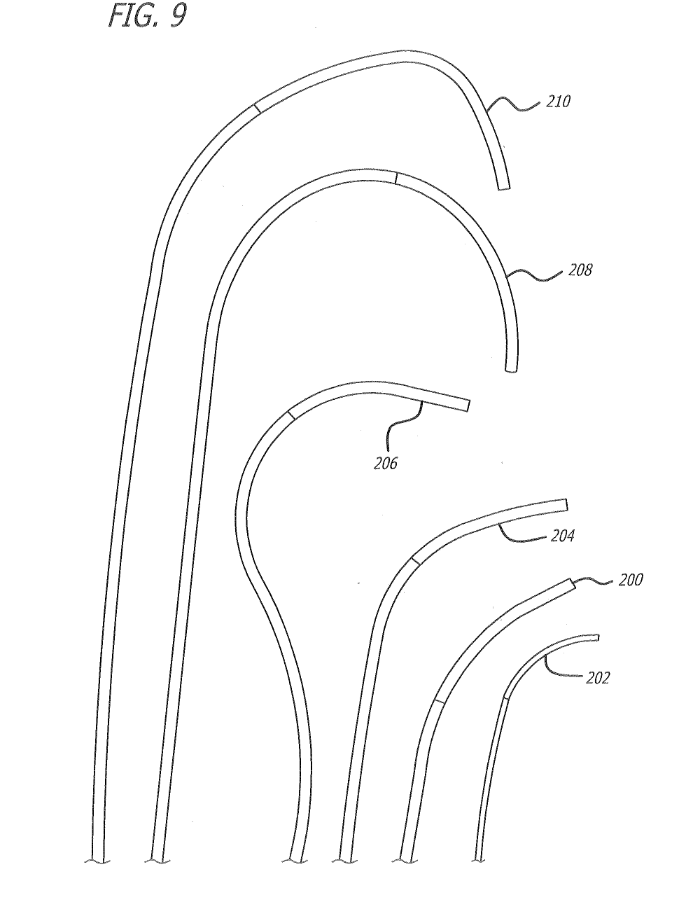

FIG. 9 illustrates several example curve shapes for comparison purposes. Catheters 200, 202 and 204 have relatively moderate distal curve shapes for accessing the coronary sinus. The catheter 206 includes two curves opposing each other in direction to form a question mark shape. The catheters 208 and 210 includes more aggressive curves in which the distal end curves around nearly 180 degrees to provide even deeper access to the coronary sinus.

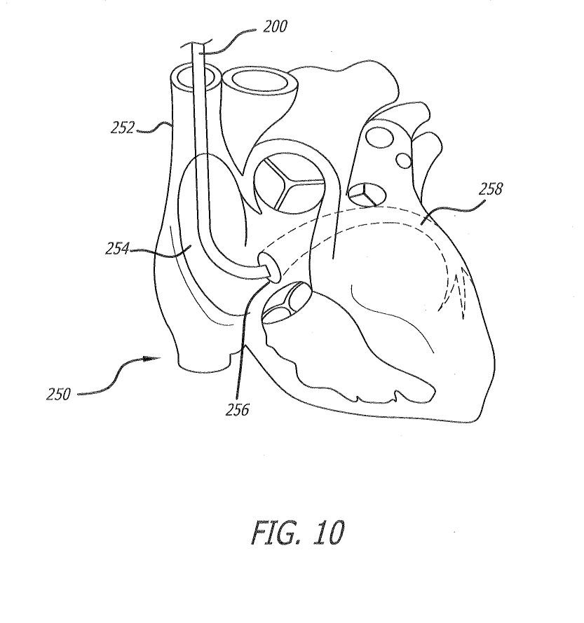

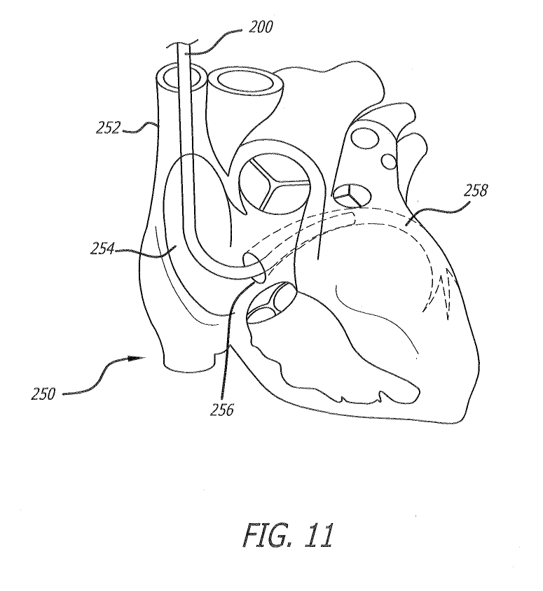

As seen in FIG. 10, the diagnostic catheter 200 can be positioned through a superior vena cava 252, into a right atrium 254 of a heart 250. The curve of the diagnostic catheter 200 allows a user to torque its distal end towards the ostium 256 of the coronary sinus 258 (e.g., with the help of a guidewire). FIG. 11 illustrates the diagnostic catheter 200 advancing into the coronary sinus. The relative flexibility of the catheter 200 allows its distal tip to be more easily advance into the coronary sinus 258 while minimizing trauma to the patient.

It should be noted that the catheters 100, 140, 150, 160, 170, 200, 202, 204, 206, 208 and 210 can be used with a variety of different procedures that require visualization techniques, such as neural procedures, mitral annuloplasty procedures or other procedures that involve access to the coronary sinus or more generally the heart. It should be further noted that any of the features of the different embodiments described in this specification may be combined with features shown on other embodiments.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A diagnostic catheter for delivering contrast within a patient comprising:
an elongated body;
a first lumen within said elongated body sized to accept a guidewire;
a second lumen within said elongated body having an opening at a proximal end of said elongated body for accepting contrast media and a second opening between said proximal end and a distal end of said elongated body for disbursing said contrast media; and
a plurality radiopaque markers disposed in said elongated body and positioned at preset distances from each other so as to visually convey measurement of a distance to a user.

2. The diagnostic catheter of claim 1, wherein said distal end of said elongated body includes a tapered region.

3. The diagnostic catheter of claim 2, wherein said second opening of said second lumen is positioned within said tapered region.

4. The diagnostic catheter of claim 1, further comprising a plurality of diffusion openings disposed along a length of said elongated body.

5. The diagnostic catheter of claim 1, wherein said second lumen is coaxial to said first lumen.

6. The diagnostic catheter of claim 1, wherein said second lumen is adjacent to said first lumen.

7. The diagnostic catheter of claim 6, further comprising a third lumen adjacent to said first lumen.

8. The diagnostic catheter of claim 1, wherein said distal end of said elongated body is curved to facilitate entry into a coronary artery.

9. The diagnostic catheter of claim 8, wherein said distal end is more flexible than said proximal end.

10. A diagnostic catheter for delivering contrast within a patient comprising:
a catheter body;
a guidewire lumen within said catheter body, said guidewire lumen positioned along an axis of said catheter body;
a contrast lumen within said catheter body having an opening located at a proximal end of said catheter body and an opening located between said proximal end and a distal end of said catheter body; and
a plurality radiopaque measurement markers within said catheter body, said markers spaced at predetermined distances from each other to visually indicate a known distance.

11. The diagnostic catheter of claim 10, wherein said contrast lumen includes at least four passages positioned along said axis of said catheter body and in communication with said opening at said proximal end.

12. The diagnostic catheter of claim 10, wherein said distal end of said catheter body is more flexible than said proximal end of said catheter body.

13. The diagnostic catheter of claim 10, wherein said distal end of said catheter body is curved.

14. The diagnostic catheter of claim 10, wherein said catheter body is progressively more flexible towards said distal end of said catheter body.

15. A method of delivering contrast within a patient comprising:
advancing a guidewire into a vascular system of a patient until a distal end of said guidewire is positioned near a desired target location;
placing a proximal end of said guidewire within a guidewire lumen of a catheter and advancing said catheter over said guidewire;
maintaining a position of said guidewire in said vascular system;
injecting contrast into a contrast lumen of said catheter and disbursing said contrast near said target location; and
viewing said contrast and a plurality of radiopaque measurement markers of said catheter.

16. The method of claim 15, wherein said injecting contrast into a contrast lumen of said catheter and disbursing said contrast near said target location further comprises passing said contrast through a second contrast lumen of said catheter.

17. The method of claim 15, wherein said disbursing said contrast near said target location further comprises diffusing said contrast along a length of said catheter.

18. The method of claim 15, wherein advancing a guidewire into a vascular system of a patient until a distal end of said guidewire is positioned near a desired target location further comprises advancing said distal end near an ostium of a coronary sinus.

19. The method of claim 18, wherein placing a proximal end of said guidewire within a guidewire lumen of a catheter and advancing said catheter over said guidewire further comprises directing a distal end of said catheter into said coronary sinus.

20. The method of claim 19, wherein directing a distal end of said catheter into said coronary sinus further comprises directing a curved portion of said distal end of said catheter into said coronary sinus and flexing said distal end of said catheter while said catheter advances into said coronary sinus.
